(19) United States
(12) Patent Application Publication
(10) Pub. No.: US 2011/0313677 A1
(43) Pub. Date: Dec. 22, 2011

(54) METHOD FOR DETERMINING THE LIVER PERFORMANCE OF A LIVING ORGANISM BY THE MEANS OF QUANTITATIVE MEASURING THE METABOLIZATION OF SUBSTRATES

(75) Inventor: Karsten Heyne, Grossbeeren (DE)

(73) Assignee: HUMEDICS GMBH, Grossbeeren (DE)

(21) Appl. No.: 13/130,143

(22) PCT Filed: Dec. 21, 2010
(86) PCT No.: PCT/EP2010/070408

§ 371 Date: May 19, 2011

(30) Foreign Application Priority Data
Dec. 24, 2009 (DE) 10 2009 055 321.5

(51) Int. Cl. G06F 19/00; A61K 49/06; G01N 33/497; A61K 49/04

(52) U.S. Cl. 702/19; 424/9.4; 424/9.3; 73/23.3

(57) ABSTRACT

A method for determining the liver performance of a living organism, in particular a human, comprising administering at least one ¹³C labelled substrate, which is converted by the liver by releasing at least one ¹³C labelled metabolization product, and determining the amount of the at least one ¹³C labelled metabolization product in the exhalation air over a definite time interval by the means of at least one measuring device with at least one evaluation unit is disclosed. Using this method, it is possible to describe the measured initial increase of the amount of the at least one ¹³C labelled metabolization product in the exhalation air using a differential equation of first order and to determine a value Aₘₐₓ (DOBₘₐₓ) and a time constant tau of the increase of the amount of ¹³C labelled metabolization product from the solution of the differential equation of first order.

### CROSS-REFERENCE TO A RELATED APPLICATION

This application is a National Phase Patent Application of International Patent Application Number PCT/EP 2010/070408, filed on Dec. 21, 2010, which claims priority of German Patent Application Number 10 2009 055 321.5, filed on Dec. 24, 2009.

### BACKGROUND

The invention relates to a method for determining the liver performance of a living organism.

The liver is an essential organ for the functioning of a living organism, in particular of a human, since in the liver a lot of substances, as for instance medicaments are enzymatically degraded. The substance degradation is thereby essentially catalyzed by the family of the cytochromes, in particular in form of a P450-oxygenases. Thereby, it has been known for some time that different cytochromes metabolize different substances. It is also known that by measuring the concentration of the metabolized substances the functioning of the liver can be estimated.

For instance, in an article by Matsumoto et al. (Digestive Diseases Science, 1987, Vol. 32, pages 344-348) the oral administration of ¹³C-methacetin to healthy and liver-damaged patients is described, wherein the ¹³C-methacetin is converted in the liver by releasing ¹³CO₂. The determination of the ¹³CO₂ amount in the exhalation air allows thereby a statement of the degree of damage of the liver.

Braden et al. (Aliment Pharmacol. Ther., 2005, Vol. 21, pages 179-185) describes the measurement of the ¹³CO_{2/}¹²CO₂ ratio in the exhalation air of individuals, whom ¹³C-methacetin has been orally administered. Thereby, in order to determine the maximum enzymatic activity it is preferably continuously measured over a time period of 60 minutes.

This approach, however, is not sufficient for the application in the clinical practice, since in particular due to the oral administration of a ¹³C-methacetin only information can be derived, if the liver functions or eventually functions still reasonably well. Hence, no direct treatment strategy for the doctor can directly be derived.

Furthermore, until now applied methods in the liver diagnostics are not individual specific, but rather allow solely statistical statements over the plurality of patients. This means that by the means of the mentioned measurements statements can be made, if the specific measuring result increases or does not increase the probability for a negative diagnostic finding. Furthermore, it is not possible to conclude from the individual measurements directly to the liver performance.

It is therefore desirably to develop simple tests which allow for prognostic statements relating to the functional resources of the liver cell tissue. Conventional laboratory parameters are not sensitive enough in order to evaluate the complex biological processes in the liver as well as its changes during disease in a reliable manner.

An analytical method which allows a quantitative determination of the liver function is described in WO 2007/000145 A2. The method is based on a substrate inundation of a substrate to be metabolized in the liver and the determination of the maximum conversion rate of the substrate, which allows for statements of the liver function capacity of a patient.

A method which allows an individual statement of the quantitative metabolization performance of an individual organ, in particular the liver can comprise different embodiments with the following properties:
1.) The dynamic of the metabolization of a substrate in the liver of a patient is determined in real time and high resolution. It can thereby be provided that the initialization of the metabolization occurs fast compared to the increase of the metabolization, i.e. it is desirable that 70% of the initialization of the metabolization occur at least two times faster.
2.) The metabolization is measured directly, i.e. that either a metabolization product is accessible directly to a measurement or another value, which is in fixed proportionality to the metabolization product, can be measured directly. This means that for instance in case of breathing gas tests, preferably each breath, but at least two breaths per minute are measured. Therefore, an intermediate storage of the breathing gas sample or a partly removal from the breathing gas is avoided in view of the procedural errors which might occur.
3.) The measured value is not changed by about more than 20% by physiological factors, i.e. the lower the influence of physiological factors is, as for instance the distribution of the substrate in the body via the blood, the more exact is the quantitative determination of the metabolization product.
4.) The metabolization process of the administered substrate is distinct and takes place exclusively or over 90% in the liver cells and nowhere else in the body.
5.) The metabolization process does not differ in its reaction efficiency from human to human, since this would counteract an individual quantitative determination. Therefore, metabolization processes are excluded which have a strong genetic variation. If at all in case of a genetic variation of the metabolization process at least the magnitude of the variation of a genetically unchanged metabolization process should be known.
6.) It is mostly desired, if the metabolization process occurs via liver enzymes or liver coenzymes, which are evenly distributed in all liver cells of the liver. If there is an accumulation of liver enzymes or liver coenzymes in specific areas of the liver, then at most only a statement of the liver performance can be made for these portions. Furthermore, the liver enzymes or liver coenzymes cannot be stressed so strongly by other metabolization reactions, so that this would lead to a change of the metabolization process in a scale of more than 30% and would lead therefore to a change of the metabolization dynamics of more than 30%.

It is not possible by using the currently known methods to realize the mentioned points.

### SUMMARY

The object of an aspect of the present invention is therefore to provide a method which allows for an individual statement of the quantitative metabolization performance of the liver.

This object is being solved by the present method for determining the liver performance of a living organism, in particular of the liver performance of a human.

Thereby, the method according to an aspect of the invention comprises the steps of administering at least one ¹³C labelled substrate, which is converted by the liver by releasing at least one ¹³C labelled metabolization product, in particular ¹³CO₂ and the step of determining the amount of the at least one formed ¹³C labelled metabolization product, in particular of the ¹³CO₂ amount, in the exhalation air over a definite time interval by the means of at least one measuring device with at least one evaluation unit. In an embodiment, the amount of the formed ¹³C labelled metabolization product, in particular of ¹³CO₂ in the exhalation air, is proportional to the amount of the at least one administered substrate. The method according to an aspect of the invention is characterized in that it is now possible based on the determined measure points to describe the measured initial increase of the amount of the at least one ¹³C labelled metabolization product, in particular of the ¹³CO₂ amount, in the exhalation air by the means of a differential equation of first order. Based on the solution of this differential equation of first order subsequently a maximum value Aₘₐₓ (also designated as DOBₘₐₓ, whereby DOB stands for “delta over baseline”) and a time constant tau of the increase of the amount of the ¹³C labelled metabolization product, in particular of the ¹³CO₂ amount, are determined.

The maximum value Aₘₐₓ or DOBₘₐₓ corresponds thereby to the maximum of the metabolization dynamics and the time constant tau corresponds to the time constant of the increase of the metabolization dynamics. In an aspect, the invention allows for the adaptation (so called fitting) of a curve to the actual measured values of the temporary changes of the ¹³C amount, wherein this curve presents a solution of the differential equation of first order and has at least two values, namely, the maximum value Aₘₐₓ and the time constant τ (tau). The solution of the differential equation is in particular an exponential function, which approximately describes the initial increase of the amount of the at least one ¹³C labelled metabolization product in the exhalation air. Its values Aₘₐₓ and tau are characteristic parameters, which characterize the initial behaviour of the increase. Therefore, an aspect of the present invention allows for an in particular defined and high resolution analysis of clinical pictures of the liver by determining two parameters of the measured initial increase. The analysis of the parameter tau and the maximum value allows in particular for such a highly defined evaluation. An aspect of the present invention provides therefore the medical doctor with improved original data for a diagnosis.

The substrate to be metabolized is transported into the liver cells. The differential equation, with which the transport of the substances reaches the liver cells, can be described by the following equation

$$\frac{d}{d t} X = f ( X , Y , Z , … ) + C \frac{{\partial }^{2}}{\partial {z}^{2}} X$$

or in three dimensions

$$\frac{d}{d t} X = f ( X , Y , Z , … ) + C Δ X$$

wherein X describes the concentration of the substrate to be metabolized and C describes the diffusion coefficient.

The diffusion coefficient C is presumed to be in a first approximation as being independent on the location. Since during evaluation of the metabolization dynamics no location specific resolution can be carried out or it is not averaged over all locations, the location dependency is reduced to the apparent diffusion constant Cₐᵥₑ and the following equation is obtained:

$$\frac{d}{d t} X = f ( X , Y , Z , … ) - {C}_{ave} X$$

It is essential that the metabolization step at the enzyme continues fast compared to the diffusion dynamic, i.e. at least as twice as fast. Thus, the metabolization for instance by the cytochrom CYP P450 1A2 takes place on average in the range of sub milliseconds.

Due to the metabolization of the substrate the substrate is being taken up by the liver, thereby the substrate concentration X is decreased and a concentration gradient is being maintained between the cell interior and cell exterior until the substance is completely degraded.

Factors on a longer time scale are provided by the function f(X, Y, Z . . . ). These influencing factors have to be less than 20% of the metabolization dynamics at the beginning of the metabolization dynamics, so that the differential equation (DE) with a DE of first order can be described according to the following equation:

$$\frac{d}{d t} X = - {C}_{ave} X$$

The solution of this DE corresponds to the equation

X(t)=X₀exp(−t/Cₐᵥₑ),

wherein Cₐᵥₑ describes a time constant tau of the conversion and X describes the concentration of the administered substrate.

The time point t=0 results from the adaptation of the dynamics or the initiation of the metabolization. If a ¹³C labelled metabolization product, as for instance ¹³CO₂, is determined, then the increase of the concentration of the metabolization product A is proportional to the decrease of the administered substrate X. Through this, the exponential falling progression of the substrate turns into an exponential increasing progression of the metabolization product according to

y(t)=Aₘₐₓ−A·exp(−t/tau),

wherein Aₘₐₓ is the maximum amplitude of the fitted function and stands therefore for the maximum concentration or amount of the metabolization product and tau is the time constant of the conversion. Thus, an exponential curve is present, which describes the increase.

In a further embodiment the solution of the differential equation of first order corresponds thus to the equation

$$y ( t ) = {A}_{max} - {A}_{0} exp ( - \frac{t - {t}_{0}}{tau} )$$

wherein (t) stands for the metabolization dynamic of the at least one substrate, t for the measuring time, t₀ for the start of the metabolization, tau for the time constant of the conversion and Aₘₐₓ for the maximum amplitude of the fitted function or the maximum concentration of the metabolization product and A₀ for the initial concentration of the metabolization product. Therefore, a determination of Aₘₐₓ and the time constant tau is possible based on the above equation.

In an embodiment, the mentioned exponential function is thus adapted to the values of the initial increase of the amount of the at least one ¹³C labelled metabolization product in the exhalation air. Subsequently, the maximum value Aₘₐₓ and the time constant tau are deduced from the adaptation.

For determining the quantitative liver performance of a living organism it is thereby of importance that the value Aₘₐₓ is proportional to the number of the liver cells involved in the metabolization and that the time constant tau provides information of the accessibility of the substance to be metabolized to the liver enzymes or liver coenzymes.

In an embodiment of the present invention, the increase of the ¹³C labelled metabolization product, in particular, the ¹³CO₂ increase, in the exhalation air is described up to a value of 70% of the maximum value of the ¹³C labelled metabolization product, in particular of the ¹³CO₂ increase, in particular up to the maximum value of the ¹³C labelled metabolization product, in particular of the ¹³CO₂ increase, by a differential equation of first order.

In a further embodiment of the invention it is now possible based on the value Aₘₐₓ or DOBₘₐₓ to determine the conversion maximum of the at least one substrate in the liver by the following equation:

$$LiMAx = \frac{{DOB}_{max} {R}_{PBD} PM}{BW}$$

wherein R_{PDB} corresponds to the value 0,011237 (Pee-Dee-Belemnite-standard of the ¹³CO_{2/}¹²CO₂-ratio), P to the CO₂ production rate, M to the molar mass of the administered substance and BW to the body weight of the person.

When applying the method according to an aspect of the invention for determining the liver performance it has to be considered that in case of a large time constant tau the directly readable maximum of the metabolization process or the metabolization dynamics can deviate from the maximum Aₘₐₓ or DOBₘₐₓ determined from the differential equation of first order. This is based on the fact that during a slow increase of the metabolization rate the influence of other factors like for instance the distribution of the substrate in the body can increase. Therefore, it is desirable to initiate the metabolization quickly, what can be for instance done by the intravenous administration of the substrate to be metabolized. The intravenous administration of the substrate guarantees a fast supply of the substrate into the liver and the fast initiation of the metabolization of the substrate connected therewith. The intravenous administration allows also for supplying a sufficiently high substrate gradient between the liver cells and the blood, which allows for the start of a metabolization dynamics and obtaining a maximum turnover rate of the substrate.

It is furthermore possible that the substrate to be metabolized contains structural units which correspond to the structures shown in FIG. 1. A compound should be in particular used as ¹³C labelled substrate which allows for the release of ¹³CO₂ by the means of a dealkylating reaction of an alkoxy group R1, in particular of a methoxy group. In general, the used substrates can be large or small molecules which either comprise a six-membered ring of carbon atoms or carbon isotopes and an alkoxy group, wherein the alkoxy group is at first hydroxylated by the P450-cytochromes present in the liver, wherein subsequently ¹³CO₂ is separated. Examples for suitable substrates are amongst others ¹³C-methacetin, phenancetin, ethoxycoumarin, caffeine, erythromycin and/or aminopyrine. It is thereby also conceivable that a carbon atom can be replaced by another atom like for instance nitrogen or sulphur. It is also conceivable that the used substrates are based on compounds with a five-membered ring, which is substituted by at least one alkoxy group R1. In this case, of course also one or two carbon atoms of the five-membered ring can be replaced by other atoms like for instance nitrogen or sulphur. It is also of course possible that the used substrate can contain different substituents. Thus, the moieties R2, R3, R4, R5 and R6 shown in FIG. 1 can be selected from a group containing halogens, alkyl groups, carboxyl groups, ether groups or silane groups. This list of possible substituents is of course not final, but can also extend to substituents known for the person skilled in the art.

In an embodiment, the ¹³C labelled substrate is administered in a concentration between 0.1 and 10 mg/kg body weight. The concentration of the substrate to be metabolized should be thereby selected such that the metabolization dynamics in the linear range is distant from the saturation. If the substrate concentration exceeds a specific value it is no longer possible to describe the increase of the amount of the ¹³C labelled metabolization product, in particular the ¹³CO₂ increase in the exhalation air by the means of a differential equation of first order. Thus, the administered amount should not be over 10 mg/kg body weight when using ¹³C-methacetin as substrate to be metabolized.

Within the present method the absolute amount of the ¹³C labelled metabolization product, in particular the ¹³CO₂ amount in the exhalation air can be determined. Thereby, the determination of the amount of the ¹³C labelled metabolization product, in particular of the ¹³CO₂ amount in the exhalation air should be carried out in real time as well as continuously. A continuous determination of the concentration of the ¹³C labelled metabolization product, in particular of the ¹³CO₂ concentration in the exhalation air in the measuring device results in the determination of more data points, through which a higher resolution and precision of the measuring curve formed by the determined data points follows. A reliable determination of the maximum value Aₘₐₓ or DOBₘₐₓ and the time constant tau should be based on at least five measuring points, in an embodiment on at least seven measuring points.

In a further embodiment the present method is combined with further analytical methods, in particular with the CT volumetry. This allows for an extensive statement of the health status of a patient and a directed operation strategy, for instance in case of occurring tumours.

In a further embodiment the present method is combined with further analytical methods, in particular magneto resonance imaging (MRI). Thereby, the ¹³C labelled substrate to be metabolized is being localized in the liver by the MRI images. The metabolization dynamics is determined by the present method and can be compared with time resolved MRI. The combination of both methods allows analysing a spatial and timely resolution of the metabolization of singular enzymes in particular in the liver. In general, the time resolution of the MRI is too slow, in order to follow a metabolization dynamics. If the data of the imaging is however synchronized with the metabolization dynamics of the present method, then an improved picture of the metabolization, for instance by grading the MRI data at different time points, can be achieved.

Additionally, the ¹³C labelled substrate to be metabolized can be selected in a variant such that they are metabolized by enzymes or coenzymes in the liver, which are not homogeneously distributed in the whole liver, but are enriched in specific regions. Through this, the metabolization performance of singular portions in the liver can be determined.

In order to determine the metabolization dynamics and the spatial illustration of this process for an enzyme or coenzyme homogeneously distributed in the liver it has to be ensured that the substrate reaches the liver cells very fast and efficient and that said substrate can be determined without distortion by the means of MRI, while simultaneously the metabolization dynamics is measured by the means of the present method.

An embodiment is the ¹³C labelled methacetin, which can be dissolved in an aqueous solution by the means of the solubiliser propylene glycol in a sufficient high concentration. The concentration of the propylene glycol is 10 to 100 mg/ml, wherein a methacetin solution with a concentration of 0.2 to 0.6% methacetin can be obtained. This specific combination of ¹³C labelled substrate (methacetin) and the solubiliser propylene glycol in aqueous solution allows for almost background free MRI measurement of the ¹³C labelled methacetin. The natural isotopic ratio of ¹³C can influence the MRI measurements in a strongly negative manner. All carbon atoms of the methacetin, the solubiliser and the remaining organic substances in the liver cells can contribute to a strongly disturbed background signal. Due to the specific selection of a ¹³C label at the methyl group bound via an ether group in methacetin (namely the methoxy group) the isotopic shift of the ¹³C labelled carbon in methacetin differs from the MRI signals of the carbon atoms of the solubiliser and the amino acid and therefore from the most other organic substances in the liver cells. Other positions of the ¹³C labelling do not show this property and prevent therefore usable MRI measurements. The contrast of the MRI imaging can be increased by a clever selection of the pulses by using coupling effects (for instance NOE, DEPT etc.).

In particular in case of very bad liver performances the combination of both methods offers significant synergetic effects. Additionally, the combination allows for a spatial resolution of the micro circulation in the liver.

The values Aₘₐₓ and tau determined by the means of the present method can be used for a multitude of applications. Following usages and applications are thereby of special importance: determining the liver performance, following the liver generation after an operation, planning operations, in particular of a damaged liver, determining the function of a transplanted liver, evaluating sepsis, in particular of intensive care patients, determining the liver damage by medication during drug approval, following long-time damages of the liver, determining liver damages by genetically modified food, in the area of operational safety in the chemical industry, occupational health care, preventive medical check-up for liver cancer, surveillance of liver diseases, adjusting the dosage of medication, determining liver damages in animals, in the environmental medicine and routine examination of the liver function.

Aspects of the present invention shall be explained in the following by the means of the following examples taking reference to the Figures without these explanations having a limiting effect to the scope of protection of the invention.

### BRIEF DESCRIPTION OF THE DRAWINGS

shows FIG. 1 a schematic illustration of the substances suitable for conducting the method;

FIG. 2 shows a schematic illustration of the course of the measuring method according to an aspect of the invention;

FIG. 3 shows a graphic illustration of the slope kinetics by the means of the measured DOB values over the measuring time:

FIG. 4a shows a graphic illustration of the slope kinetics in case of a normal liver performance;

FIG. 4b shows a graphic illustration of the slope kinetics in case of cirrhosis of the liver;

FIG. 4c shows a graphic illustration of the slope kinetics in case of heavy liver damages;

FIG. 4d shows a graphic illustration of the slope kinetics in case of liver failure;

FIG. 5 shows a graphic illustration of the conversion maximum LiMAx via the time in case of normal liver performance, reduced liver performance and liver failure:

FIG. 6 shows a schematic illustration of the transport of an administered substance into the liver;

FIG. 7 shows a graphic illustration of the slope kinetics for determining the data of the maximum value A and the time constant tau; and

FIG. 8 shows a graphic illustration of the decrease of the concentration of a substrate to be metabolized and the increase of concentration of a metabolization product in the blood.

### DETAILED DESCRIPTION

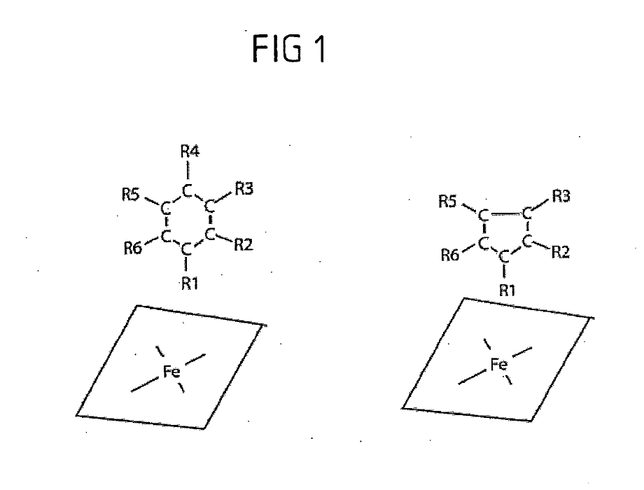

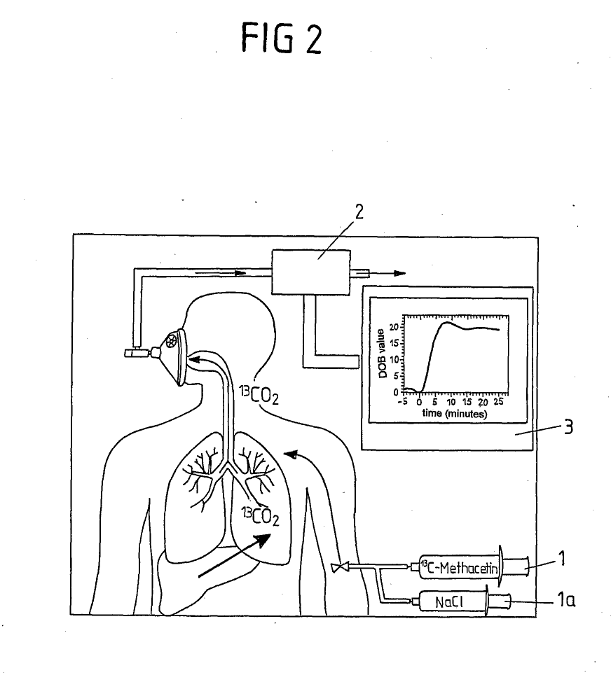

In an embodiment of the present method the determination of the liver performance of a human occurs according to a scheme as shown in FIG. 2. During this measurement course the metabolization is started by the intravenous administering of the substrate to be metabolized, in particular ¹³C methacetin 1 in combination with an isotonic sodium chloride solution 1a.

Due to the intravenous administration the fast substrate inundation and the fast initiation of the substrate metabolization, which is required for the analysis, is guaranteed. The initiation of the substrate metabolization caused by the enzymatic conversion of the substrate in the liver is thereby faster than the breathing rhythm.

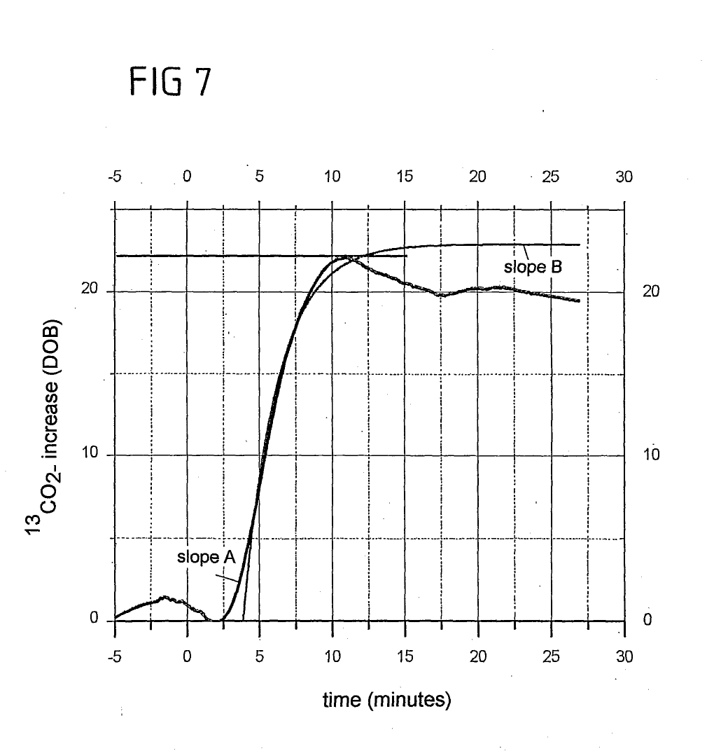

The transport of the administered substrate into the liver and the conversion or degradation of the substrate taking place there is schematically clarified in FIG. 7. The administered substrate (double cross-hatched circles) as for instance ¹³C methacetin is transported by a specific transport constant into the liver cells, is there converted by the respective enzymes (single cross-hatched six membered hexagons), in particular P450 oxygenases, for instance by the means of dealkylation with a specific reaction constant and the dealkylated product (single cross hatched circles), for instance Paracetamol is transported with a specific transport constant and the ¹³C labelled metabolization product (single cross hatched circle) for instance ¹³CO₂ with a specific transport constant out of the liver cells into the blood.

Beside an enzymatic activation of the substrate in particular by the P450 oxygenases also a release or activation of the substrate by the means of radiation or other fast processes is conceivable. The released metabolization product for instance ¹³CO₂ is transported via the blood into the lung and is there exhaled. The exhalation air is continuously transported into the measuring device 2, e.g., via a breathing mask and a connecting tube and is analyzed by the means of a computer 3 (Stockmann et al., Annals of Surgery, 2009, 250: col. 119-125). A measuring device suitable for the present method is for instance described in WO 2007/107366 A1.

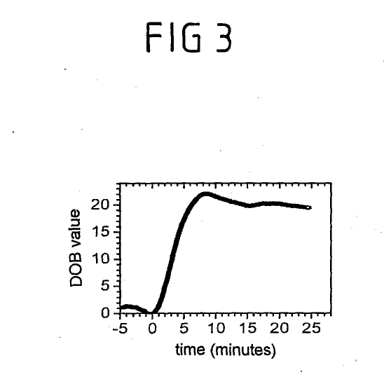

Due to the specific measuring device being applied it is possible to follow the metabolization of the substrate in each breath in real time. This is emphasized in FIG. 3. The diagram of FIG. 3 shows an increase of the ¹³CO₂ concentration by the way of the DOB value in the exhalation air wherein the increase corresponds to a differential equation of first order. Thereby 1 DOB indicates a change of the ¹³CO₂ to ¹²CO₂ ratio at about thousandth part over the natural ratio. As described before, Aₘₐₓ or DOBₘₐₓ as well as the time constant tau are deducible from said slope. After the ¹³CO₂ increase has reached a maximum a decrease of the ¹³CO₂ concentration occurs what can be attributed to further dynamic processes in the body which contribute to the degradation of the measured signal.

described metabolization dynamics it is possible to follow directly and immediately the metabolization of the administered substrate by the enzymes present in the liver. If methacetin is administered as substrate, it is demethylated by the enzyme CYP1A2. When analysing the slope kinetics of the administered methacetin which corresponds to a differential equation of first order and the parameters Aₘₐₓ and tau derived from it, it is now possible to directly determine the liver performance. Thereby the maximum value Aₘₐₓ allows a statement about the number of healthy liver cells and the liver volume being available for metabolization, while the slope in form of the time constant tau allows statements of the access rate of the substrate into the liver cell. Thus, in particular, the time constant tau allows statements if the liver is actually able to take up the substrate.

FIG. 7 shows by the means of an example the determination of the relevant parameters on the basis of a curve, which illustrates the increase of the ¹³CO2 in the breathing air after taking ¹³C labelled methacetin, see here also the explanations to FIG. 3. Based on the determined data points (curve A) with a measured maximum value A of 22,01 DOB an adaptation (fitting) with one solution of a differential equation of first order (curve B) is carried out as described above. Based on the solution of the differential equation according to

$$y ( t ) = {A}_{max} - {A}_{0} exp ( - \frac{t - {t}_{0}}{tau} )$$

the determination of the amplitude Aₘₐₓ of the fitted function with 22,09 DOB and a time constant tau for the conversion of 2.42 minutes occurs. A small time constant of 2.42 minutes indicates thereby a good liver permeability while a slow increase of a curve based on the measuring points indicates time constants in the area of over five minutes and therefore a hardening of the liver tissue and the worsened liver permeability connected therewith.

Beside or additionally to the determination of the amount of a ¹³C labelled metabolization product as for instance ¹³CO₂ in the exhalation air for estimating the liver performance it is also conceivable to follow the concentration decrease of the dealkylated product in the blood and to deduce from the corresponding slope kinetics a time constant tau.

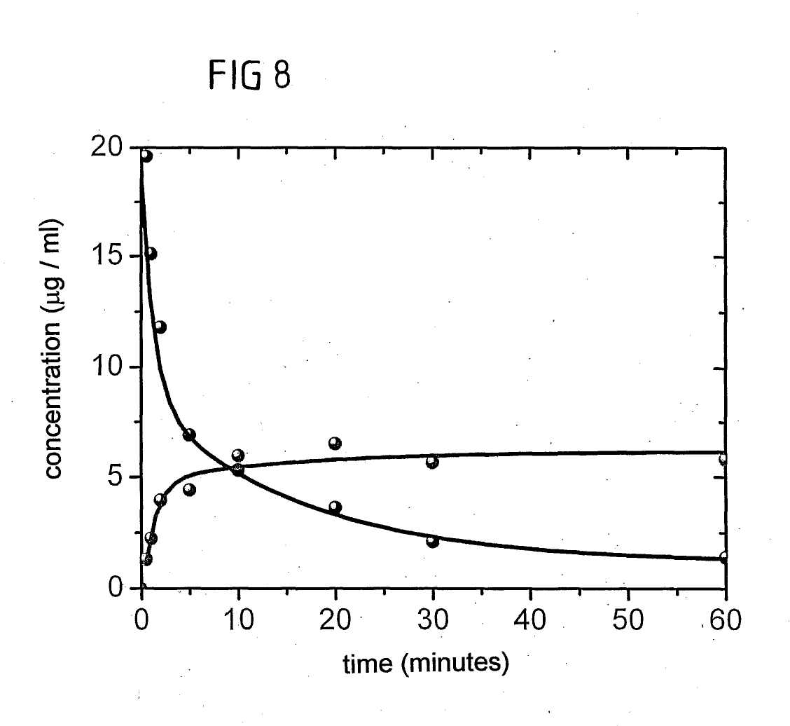

This method variant is shown in FIG. 8. The concentration changes of the administered ¹³C labelled substrate, for instance ¹³C methacetin and of the dealkylation product formed in the liver, for instance Paracetamol, are followed by the means of a suitable analytical method, for instance HPLC. The concentration of the ¹³C methacetin decreases due to the metabolization (exponentially decreasing curve starts at an initial concentration of 20 μg/ml ¹³C methacetin) while the concentration of the Paracetamol increases in return (lower curve in FIG. 8). The initial concentration changes can also be described here with a differential equation of first order. By the means of the described solution for a differential equation of first order the respective time constants are deducible, wherein the time constant τ1 for the initial fast concentration increase of the Paracetamol is 1.3 min while the time constant τ2 for the subsequent decelerated concentration increase due to a further distribution in the blood is 16 min.

The present method for determining the liver performance is applicable for a multitude of usages.

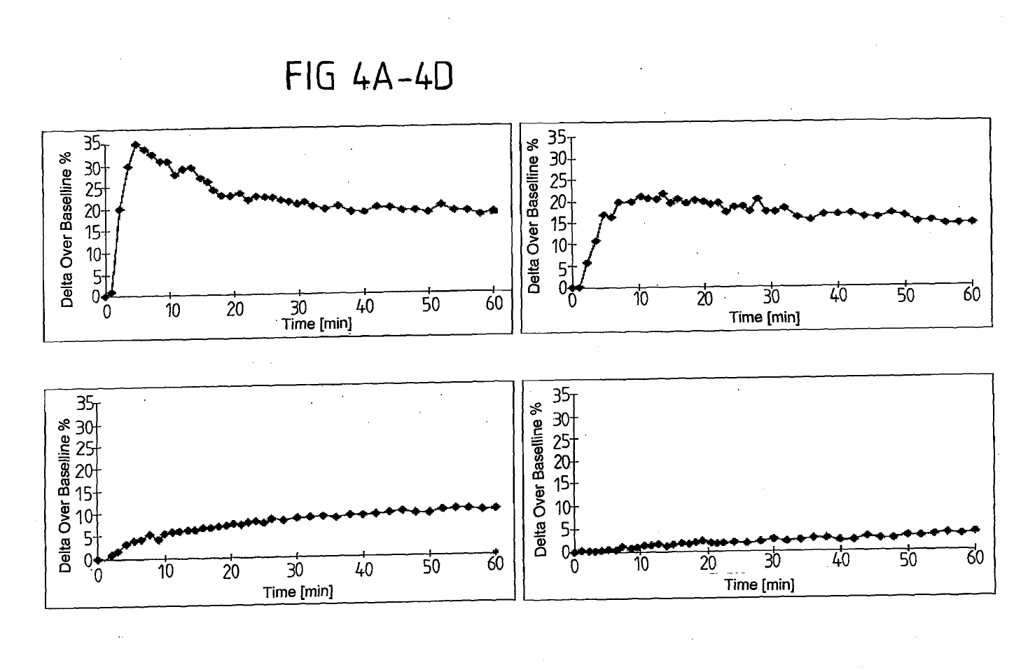

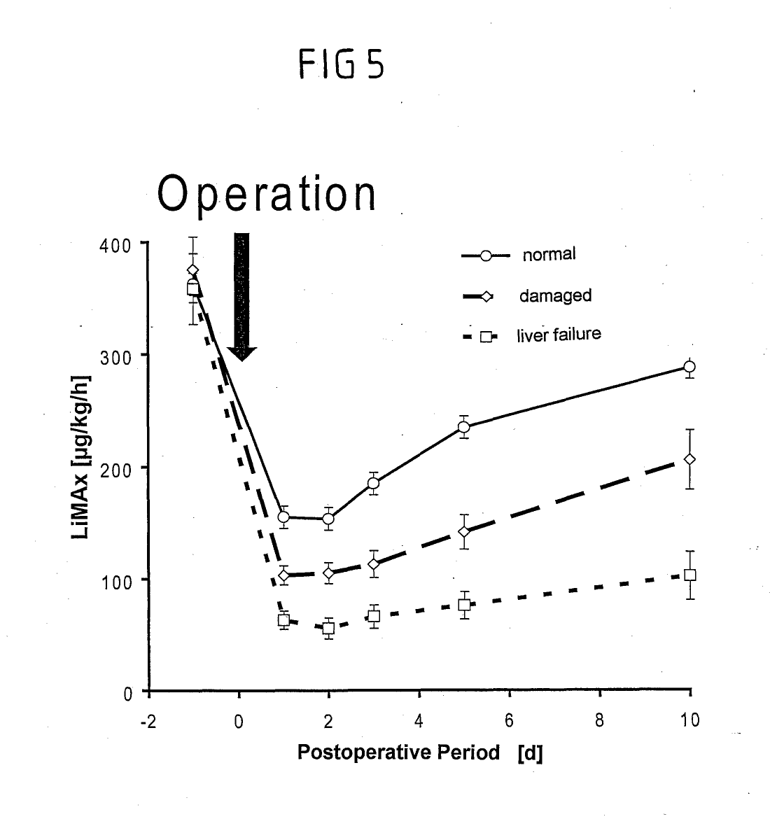

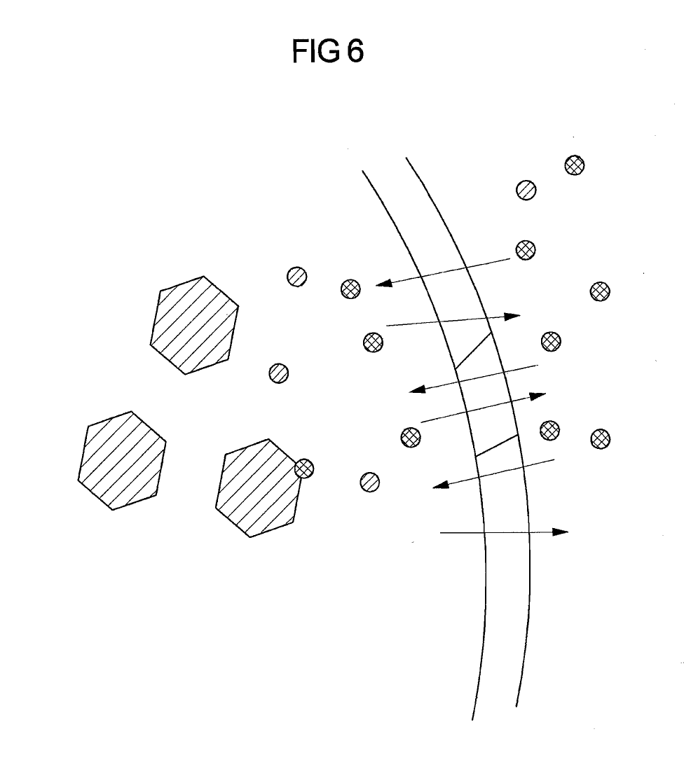

Thus, the method allows an estimation of the general health status of a patient, in particular an estimation of the liver performance of a patient. In FIGS. 4a-d the increase of the metabolization is shown as function of time. Thereby, different slope kinetics are obtained for different clinical pictures with different maximum values A and different time constants τ. As described, the value A allows the determination of the maximum conversion LiMAx which is directly proportional to the liver performance. FIG. 4a shows a normal liver performance with a maximum conversion LiMAx of 504 μg/h/kg while in FIGS. 4b-4d different clinical pictures are emphasized. In case of cirrhosis of the liver, the metabolization of the administered substrate is reduced so that the maximum conversion LiMAx only reaches a value of 307 μg/h/kg. In case of further liver damages up to a liver failure the maximum conversion of the administered substrate is reduced accordingly to a value of 144 μg/h/kg (FIG. 4c) or 55 μg/h/kg (FIG. 4d).

The present method allows also the prediction or tracing of the liver generation and examination of the liver status after an operation as for instance after a liver resection. Thus, it is possible by the means of the present method to examine already a few minutes after a liver operation or even already during the operation if and to which extend the liver is efficient.

In FIG. 5 the liver performances after a liver operation are shown. The maximum conversion LiMAx differs significantly between a healthy regular liver, a weakened liver or a strongly damaged liver. It usually takes a few days after an operation until the liver is regenerated. If the maximum conversion LiMAx and therefore, the liver performance after an operation has already been very low it can be predicted that the liver of the patient won't recover and the patient will die with high probability. By the means of the present method, however, a fast recognition of such critical cases is possible so that the affected patients can be alternatively treated for instance by a liver transplantation and can be rescued thereby.

The present method allows also a prediction of the operation result before an operation and therefore a suitable operation planning. Thus, for instance in combination with a CT volumetry not only the damaged tissue as for instance tumour tissue, but also the tissue which has to necessarily be removed can be determined before a liver operation. This is necessary since in case of a tumour treatment as much tissue around the tumour as possible has to be removed in order to minimize the risk of spreading of a tumour. If thereby, however, too much liver volume is removed, the possibility exists that the patient deceases. The size of the liver volume to be removed depends on the liver performance of the remaining liver volume. Due to the exact determination of the liver performance of the existing liver volume an operation can be planned with utmost precision so that the patient has optimal chances for surviving and regenerating.

This is shown by the means of the following example. If the tumour volume is for instance 153 ml then it is reasonable to remove a total of ca. 599 ml liver volume. In case of a total liver volume of 1450 ml thus a residual volume of 698 ml would remain what would ensure a survival of the patient. The maximum conversion LiMAx of the administered ¹³C methacetin is before the operation 307 μg/h/kg. The aspired residual volume of 698 ml would correspond to a maximum conversion LiMAx of 165 μg/h/kg. The conversion can continuously be determined already during the operation by the means of the present method so that it is guaranteed that the residual volume of 698 ml required for survival is reached. In the present case the residual volume of the liver after the operation is 625 ml and has a maximum conversion of 169 μg/h/kg. Due to a direct comparison of the healthy liver volume with the LiMAx value the liver volume to be resized can be determined via the rule of three in order to obtain an aimed LiMAx value.

The present method allows also for the determination of the function or the post operative non-function (PNS) of a transplanted liver. In about 5% of the cases it happens after a liver transplantation that the transplanted liver for instance due to an insufficient blood circulation does not function. Until now, this can only be detected after several days. By the means of the present method it is however possible to detect the malfunction of the liver already after a few minutes since the time constant τ provides information about the accessibility of the administered substrate to the liver. The patient can be treated accordingly and for instance a new transplantation can be carried out.

The measurement of the operational success after a liver transplantation and the planning of further treatment steps are possible by the means of the present method. Thus, after a liver transplantation the performance of the liver can be determined immediately and directly by the present method and the further treatment of the patient can be optimized individually.

The present method allows furthermore the evaluation of the risk of sepsis for intensive care patients. It is known that the risk to die due to a sepsis is very high in the intensive care medicine. It is now possible by the means of the present measuring method to determine directly during admission and treatment a liver damage or a normal function of the liver cells.

The determination of the liver damage is also of importance in particular during approval of medicaments and drugs. Therefore, one of the most important applications of the present method is the use of the method for examining liver damages caused by medicaments and drugs in the course of a drug approval. During the drug approval it has to be shown in a toxicology test that the drugs to be approved do not damage the liver. Such risk estimation is usually deduced from a series of different animal tests. However, unexpected side effects occur often in humans, which are only difficult to detect in animal tests. In contrast, by the means of the present method a toxic effect to animals and humans can be determined exactly and quantitatively. Due to the present method which allows for a reliable quantitative determination of the liver performance it is now possible to carry out tests for drug dosages faster and more exactly.

Long term damages combined with a rearrangement of the liver caused by medicaments as for instance contraceptives, can also be followed by the means of the present method. If medicaments are taken regularly, as for instance in case of contraceptives, changes of the liver can occur which influence at first the accessibility of the liver cells and cause later a reduction of the liver performance. These changes of the liver can be determined by the slope times τ, via which the access rate of the substance into the liver cells can be determined and the maximum value A, which allows statements about the number of healthy liver cells. Regular tests with the present measuring method allow therefore the detection of such liver changes. Based on the determined data the doctor can carry out a change of administering the medicament so that no further liver changes occur.

The influence of genetically modified substances and food on living organisms, in particular human, is currently only difficult to detect. This is in particular due to the fact that the concentration of harmful biological substance is often below or just under the detection limit or the harmfulness of said substance is not known until now. The present method allows the clear detection of the damaging of the liver by genetically modified food.

Influences of chemicals in the chemical industry or the pharmaceutical industry can also be followed, monitored and identified by the means of the present method. This allows for a reliable examination of the human health in the working place.

Further applications of the present method are in the area of occupational medicine for estimating health risks, in screening liver cancer, monitoring liver illnesses, as for instance hepatitis, detecting liver damages in animals as for instance caused by the plant Senecio jacobaea I. in horses, poisoning and in the environmental medicine in the search for live damaging substances in soil, food and/or drinking water.

A well suited application of the present method is the adjustment of medicaments. Since the liver metabolizes the plurality of all administered drugs, a majority of the drugs is accordingly metabolized in case of a high liver performance; while in case of a bad liver performance a low amount of the drugs is metabolized. This however means for a patient that depending on liver performance the dosage of the drugs in the body is different and can therefore also unfold a different effectiveness. Therefore, an optimal effect of the drug should be adapted to the liver performance. As an example the administration of Tacrolimus, an immunosuppressant against rejection reactions after organ transplantation is being pointed out. The exact adjustment of the dosage of Tacrolimus is of high importance since a high dosage of Tacrolimus is toxic and if the dosage is too small it has no effect. If the liver performance is now exactly known, the dosage can be adjusted exactly and the effect of the drug can be optimized.

The present method can also be used by a family doctor for liver check-ups due to its simplicity and fastness in order to request the liver performance as part of the health status.

1. A method for determining the liver performance of a living organism, in particular a human, comprising;
administering at least one ¹³C labelled substrate, which is converted by the liver by releasing at least one ¹³C labelled metabolization product, and
determining the amount of the at least one ¹³C labelled metabolization product in the exhalation air over a definite time interval by the means of at least one measuring device with at least one evaluation unit,
wherein
the measured initial increase of the amount of the at least one ¹³C labelled metabolization product in the exhalation air is described by a differential equation of first order and the value Aₘₐₓ for the maximum concentration of the ¹³C labelled metabolization product and the time constant tau of the increase of the amount of the ¹³C labelled metabolization product are determined from the solution of the differential equation of first order,

2. The method according to claim 1, wherein the at least one ¹³C labelled metabolization product in the exhalation air is ¹³CO₂.

3. The method according to claim 1, wherein the ¹³CO₂ increase of the ¹³C metabolization product in the exhalation air is described up to a value of 70% of the maximum value of the ¹³C labelled metabolization product, in particular up to the maximum value of the ¹³C labelled metabolization product by a differential equation of first order.

4. The method according to claim 1, wherein the amount of the formed ¹³C labelled metabolization product, in particular of ¹³CO₂ is proportional to the amount of the at least one administered substrate.

5. The method according to claim 1, wherein that as solution of the differential equation of first order the equation
$$y ( t ) = {A}_{max} - {A}_{0} exp ( - \frac{t - {t}_{0}}{tau} )$$
is used, wherein y(t) describes the metabolization dynamics of the at least one substrate, Aₘₐₓ the maximum amplitude of the fitted function or the maximum concentration of the metabolization product, A₀ the initial concentration of the metabolization product, tau the time constant, t₀ the start of the metabolization and t the measuring time.

6. The method according to claim 5, wherein the said exponential function is adapted to the measured data of the initial increase of the amount of the at least one ¹³C labelled metabolization product in the exhalation air and the maximum value Aₘₐₓ and the time constant tau are determined from the adaptation.

7. The method according to claim 1, wherein based on the value Aₘₐₓ the maximum conversion of the at least one substrate in the liver is determined by the equation
$$LiMAx = \frac{{A}_{max} {R}_{PBD} PM}{BW}$$
wherein R_{PBD} as the Pee-Dee-Belemnite-Standard of the ¹³CO_{2/}¹²CO₂-ratio corresponds to the value 0,011237, P to the CO₂ production rate, M to the molar mass of the administered substance and BW to the body weight of the person.

8. The method according to claim 1, wherein the ¹³C labelled substrate is administered in a concentration between 0.1 and 10 mg/kg body weight.

9. The method according to claim 1, wherein as ¹³C labelled substrate a substrate is used from which ¹³CO₂ is released by the means of a de-alkylation reaction of an alkoxy group, in particular a methoxy group.

10. The method according to claim 1, wherein as substrate a ¹³C labelled methacetin, phenacetin, aminopyrine, caffeine, erythromycin and/or ethoxycoumarin is used.

11. The method according to claim 1, wherein the absolute amount of the ¹³C labelled metabolization product, in particular the absolute ¹³CO₂ amount, in the exhalation air is determined.

12. The method according to claim 1, wherein the determination of the formed ¹³C labelled metabolization product, in particular of ¹³CO₂, occurs in real time.

13. The method according to claim 1, wherein the amount of the formed ¹³C labelled metabolization product, in particular the ¹³CO₂ amount in the exhalation air is continuously determined by the measuring device.

14. The method according to claim 1, wherein the complete or a part of the exhalation air is continuously transferred via a breathing mask and a connecting tube to the measuring device.

15. The method according to claim 1, wherein said method is combined to other analytical methods, in particular to the CT volumetry or the magnetic resonance imaging.

16. A use of at least one substance of the group comprising ¹³C labelled methacetin, phenacetin, aminopyrine, caffeine, erythromycin and ethoxycoumarin as substrate in the method according to claim 1.

17. A use of an aqueous solution of ¹³C methacetin and propylene glycol as substrate in the method according to claim 1.

18. The use according to claim 17, wherein the concentration of said propylene glycol is 10 to 100 mg/ml and the concentration of said ¹³C methacetin is 0.2 to 0.6%.
